(19) United States
(12) Patent Application Publication
(10) Pub. No.: US 2009/0197302 A1
(43) Pub. Date: Aug. 6, 2009

(54) Method for Producing an L-Amino Acid Using a Bacterium of the Enterobacteriaceae Family Having Attenuated Expression of the nac Gene

(76) Inventors: Dmitriy Vladimirovich Filippov, Moscow (RU); Elvira Borisovna Voroshilova, Moscow (RU); Mikhail Markovich Gusyatiner, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/849,415

(22) Filed: Sep. 4, 2007

Related U.S. Application Data
(63) Continuation of application No. PCT/JP06/05190, filed on Mar. 9, 2006.
(60) Provisional application No. 60/723,923, filed on Oct. 6, 2005.

(30) Foreign Application Priority Data
Mar. 10, 2005 (RU) 2005106347

(51) Int. Cl. C12P 21/00; C12N 1/21

(52) U.S. Cl. 435/69.1; 435/252.1; 435/252.33

(57) ABSTRACT

The present invention provides a method for producing an L-amino acid using a bacterium of the Enterobacteriaceae family, particularly a bacterium belonging to the genus Escherichia or Pantoea, which has been modified to attenuate expression of the nac gene.

This application is a continuation of PCT/JP2006/305190, filed Mar. 9, 2006. This application also claims priority under 35 U.S.C. §119 to Russian application 2005106347 filed on Mar. 10, 2005, and U.S. Provisional application 60/723,923, filed on Oct. 6, 2005. Each of these documents is incorporated by reference. The Sequence Listing in electronic format filed herewith is also hereby incorporated by reference in its entirety (File Name: US-212_Seq_List_Copy_{—}1; File Size: 9 KB; Date Created: Sep. 4, 2007).

### BACKGROUND OF THE INVENTION

1. Field of Invention

The present invention relates to the microbiological industry, and specifically to a method for producing an L-amino acid using a bacterium of the Enterobacteriaceae family, which has been modified to attenuate expression of the nac gene.

2. Description of the Related Art

The Nac (Nitrogen Assimilation Control) protein belongs to the LysR family. This regulator protein participates in controlling several genes involved in histidine utilization and nitrogen assimilation. Nac represses the asnC gene and the gabDTPC operon when the supply of nitrogen is limited. In brief, nitrogen-limited cell growth leads to glutamine starvation. Through a complex cascade of events, glutamine starvation in Klebsiella aerogenes leads to phosphorylation (and activation) of the transcriptional regulator NtrC. Once phosphorylated, NtrC activates RNA polymerase carrying σ54 to transcribe a number of genes, including the nac gene, which codes for the Nac protein. The Nac protein in turn activates RNA polymerase carrying σ70 to transcribe a number of operons, resulting in the producion of proteins which supply the cell with ammonium or glutamate from alternative organic sources. The Nac protein also represses operons which assimilate ammonium when over-abundant. The operons activated by the Nac protein in K. aerogenes include hutUH, putP, and ureDABCEFG, which code for enzymes required for the catabolism of histidine, proline, and urea, respectively. The operons repressed by the Nac protein include gdhA (glutamate dehydrogenase [GDH]), gltBD (glutamate synthase), and nac itself (Schwacha, A. and Bender, R. A., J. Bacteriol., 175, 7 2107-2115 (1993)).

The nac gene encoding the Nac protein from Escherichia coli has been reported (Muse, W. B. and Bender R. A., J. Bacteriol., 180, 5 1166-1173 (1998)).

But currently, there have been no reports of inactivating the nac gene for the purpose of producing L-amino acids.

### SUMMARY OF THE INVENTION

Aspects of the present invention include enhancing the productivity of cellular strains able to produce L-amino acid, and providing a method for producing an L-amino acid using these strains.

The above aspects were achieved by finding that attenuating expression of the nac gene can enhance production of L-amino acids, such as L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, L-arginine, L-phenylalanine, L-tyrosine, and L-tryptophan.

The present invention provides a bacterium of the Enterobacteriaceae family having an increased ability to produce amino acids, such as L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, L-arginine, L-phenylalanine, L-tyrosine, and L-tryptophan.

It is an aspect of the present invention to provide an L-amino acid producing bacterium of the Enterobacteriaceae family, wherein the bacterium has been modified to attenuate expression of the nac gene.

It is a further aspect of the present invention to provide the bacterium as described above, wherein the expression of the nac gene is attenuated by inactivation of the nac gene.

It is a further aspect of the present invention to provide the bacterium as described above, wherein the bacterium belongs to the genus Escherichia.

It is a further aspect of the present invention to provide the bacterium as described above, wherein the bacterium belongs to the genus Pantoea.

It is a further aspect of the present invention to provide the bacterium as described above, wherein said L-amino acid is selected from the group consisting of an aromatic L-amino acid and a non-aromatic L-amino acid.

It is a further aspect of the present invention to provide the bacterium as described above, wherein said aromatic L-amino acid is selected from the group consisting of L-phenylalanine, L-tyrosine, and L-tryptophan.

It is a further aspect of the present invention to provide the bacterium as described above, wherein said non-aromatic L-amino acid is selected from the group consisting of L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine.

It is a further aspect of the present invention to provide a method for producing an L-amino acid comprising:

cultivating the bacterium as described above in a medium so to produce and excrete said L-amino acid into the medium, and

collecting said L-amino acid from the medium.

It is a further aspect of the present invention to provide the method as described above, wherein said L-amino acid is selected from the group consisting of an aromatic L-amino acid and a non-aromatic L-amino acids.

It is a further aspect of the present invention to provide the method as described above, wherein said aromatic L-amino acid is selected from the group consisting of L-phenylalanine, L-tyrosine, and L-tryptophan.

It is a further aspect of the present invention to provide the method as described above, wherein said non-aromatic L-amino acid is selected from the group consisting of L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine.

The present invention is described in detail below.

### BRIEF DESCRIPTION OF DRAWINGS

FIG. 1 shows the relative positions of primers nac L and nac R on plasmid pACYC184, which is used for amplification of the cat gene.

FIG. 2 shows the construction of the chromosomal DNA fragment containing the inactivated nac gene.

### DETAILED DESCRIPTION OF THE PREFERRED EMBODIMENTS

1. Bacterium of the Present Invention

The bacterium of the present invention is an L-amino acid producing bacterium of the Enterobacteriaceae family, wherein the bacterium has been modified to attenuate expression of the nac gene.

In the present invention, “L-amino acid producing bacterium” means a bacterium, which has an ability to produce and excrete an L-amino acid into a medium, when the bacterium is cultured in the medium.

The phrase “L-amino acid-producing bacterium” as used herein also means a bacterium which is able to produce and cause accumulation of an L-amino acid in a culture medium in an amount larger than a wild-type, unmodified, or parental strain of E. coli, such as E. coli K-12, and preferably means that the microorganism is able to cause accumulation in a medium of an amount not less than 0.5 g/L, more preferably not less than 1.0 g/L of the target L-amino acid. The term “L-amino acids” includes L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine.

The term “aromatic L-amino acid” includes L-phenylalanine, L-tyrosine, and L-tryptophan. The term “non-aromatic L-amino acid” includes L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine. L-threonine, L-lysine, L-cysteine, L-leucine, L-histidine, L-glutamic acid, L-phenylalanine, L-tryptophan, L-proline, and L-arginine are particularly preferred.

The Enterobacteriaceae family includes bacteria belonging to the genera Escherichia, Enterobacter, Erwinia, Klebsiella, Pantoea, Photorhabdus, Providencia, Salmonella, Serratia, Shigella, Morganella Yersinia, etc. Specifically, those classified into the Enterobacteriaceae according to the taxonomy used in the NCBI (National Center for Biotechnology Information) database (http://www.ncbi.nlm.nih.gov/Taxonomy/Browser/wwwtax.cgi?id=91347) can be used. A bacterium belonging to the genus Escherichia or Pantoea is preferred.

The phrase “a bacterium belonging to the genus Escherichia” means that the bacterium is classified into the genus Escherichia according to the classification known to a person skilled in the art of microbiology. Examples of a bacterium belonging to the genus Escherichia as used in the present invention include, but are not limited to, Escherichia coli (E. coli).

The bacterium belonging to the genus Escherichia that can be used in the present invention is not particularly limited, however for example, bacteria described by Neidhardt, F. C. et al. (Escherichia coli and Salmonella typhimurium, American Society for Microbiology, Washington D.C., 1208, Table 1) are encompassed by the present invention.

The phrase “a bacterium belonging to the genus Pantoea” means that the bacterium is classified as the genus Pantoea according to the classification known to a person skilled in the art of microbiology. Some species of Enterobacter agglomerans have been recently re-classified into Pantoea agglomerans, Pantoea ananatis, Pantoea stewartii or the like, based on nucleotide sequence analysis of 16S rRNA, etc. (Int. J. Syst. Bacteriol., 43, 162-173 (1993)).

The phrase “bacterium has been modified to attenuate expression of the nac gene” means that the bacterium has been modified in such a way that the modified bacterium contains a reduced amount of the Nac protein as compared with an unmodified bacterium, or the modified bacterium is unable to synthesize the Nac protein. The phrase “bacterium has been modified to attenuate expression of the nac gene” also means that the target gene is modified in such a way that the modified gene encodes a mutant Nac protein which has decreased activity.

The phrase “inactivation of the nac gene” means that the modified gene encodes a completely non-functional protein. It is also possible that the modified DNA region is unable to naturally express the gene due to a deletion of a part of the gene, the shifting of the reading frame of the gene, the introduction of missense/nonsense mutation(s), or the modification of an adjacent region of the gene, including sequences controlling gene expression, such as a promoter, enhancer, attenuator, ribosome-binding site, etc.

The nac (Nitrogen Assimilation Control) gene encodes the Nac protein (synonym-B1988), which is a transcriptional activator of nitrogen assimilation. The nac gene of E. coli (nucleotides complementary to nucleotides 2059040 to 2059957 in the GenBank accession number NC_{—}000913.2; gi:49175990; SEQ ID NO: 1) is located between the cbl and the erfK genes on the chromosome of E. coli K-12. The nucleotide sequence of the nac gene and the amino acid sequence of Nac encoded by the nac gene are shown in SEQ ID NO: 1 and SEQ ID NO:2, respectively.

Since there may be some differences in DNA sequences between the genera or strains of the Enterobacteriaceae family, the nac gene to be inactivated on the chromosome is not limited to the gene shown in SEQ ID No:1, but may include genes homologous to SEQ ID No:1 encoding a variant protein of the Nac protein. The phrase “variant protein” as used in the present invention means a protein which has changes in the sequence, whether they are deletions, insertions, additions, or substitutions of amino acids, but still maintains the activity of the product as the Nac protein which represses the asnC gene and the gabDTPC operon in response to nitrogen limitation. The number of changes in the variant protein depends on the position or the type of amino acid residues in the three dimensional structure of the protein. It may be 1 to 30, preferably 1 to 15, and more preferably 2 to 5 in SEQ ID NO: 2. These changes in the variants can occur in regions of the protein which are not critical for the function of the protein. This is because some amino acids have high homology to one another so the three dimensional structure or activity is not affected by such a change. These changes in the variant protein can occur in regions of the protein which are not critical for the function of the protein. Therefore, the protein variant encoded by the nac gene may have a homology of not less than 80%, preferably not less than 90%, and most preferably not less than 95%, with respect to the entire amino acid sequence shown in SEQ ID NO. 2, as long as the ability of the Nac protein to complement nac mutation prior to inactivation is maintained.

Homology between two amino acid sequences can be determined using the well-known methods, for example, the computer program BLAST 2.0, which calculates three parameters: score, identity and similarity.

Moreover, the nac gene may be a variant which hybridizes under stringent conditions with the nucleotide sequence shown in SEQ ID NO: 1, or a probe which can be prepared from the nucleotide sequence, provided that it encodes a functional Nac protein prior to inactivation. “Stringent conditions” include those under which a specific hybrid, for example, a hybrid having homology of not less than 60%, preferably not less than 70%, more preferably not less than 80%, still more preferably not less than 90%, and most preferably not less than 95%, is formed and a non-specific hybrid, for example, a hybrid having homology lower than the above, is not formed. For example, stringent conditions are exemplified by washing one time or more, preferably two or three times at a salt concentration of 1×SSC, 0.1% SDS, preferably 0.1×SSC, 0.1% SDS at 60° C. Duration of washing depends on the type of membrane used for blotting and, as a rule, should be what is recommended by the manufacturer. For example, the recommended duration of washing for the Hybond™ N+ nylon membrane (Amersham) under stringent conditions is 15 minutes. Preferably, washing may be performed 2 to 3 times. The length of the probe may be suitably selected depending on the hybridization conditions, and is usually 100 bp to 1 kbp.

Expression of the nac gene can be attenuated by introducing a mutation into the gene on the chromosome so that intracellular activity of the protein encoded by the gene is decreased as compared with an unmodified strain. Such a mutation on the gene can be replacement of one base or more to cause one or more amino acid substitutions in the protein encoded by the gene (missense mutation), introduction of a stop codon (nonsense mutation), deletion of one or two bases to cause a frame shift, insertion of a drug-resistance gene, or deletion of a part of the gene or the entire gene (J. Biol. Chem., 1997, 272 (13): 8611-8617, J. Antimicrobial Chemotherapy, 2000, 46: 793-79). Expression of the nac gene can also be attenuated by modifying an expression regulating sequence such as the promoter, the Shine-Dalgarno (SD) sequence, etc. (WO95/34672, Biotechnol. Prog. 1999, 15, 58-64).

For example, the following methods may be employed to introduce a mutation by gene recombination. A mutant gene encoding a mutant protein having a decreased activity is prepared, and a bacterium to be modified is transformed with a DNA fragment containing the mutant gene. Then the native gene on the chromosome is replaced with the mutant gene by homologous recombination, and the resulting strain is selected. Such gene replacement using homologous recombination can be conducted by the method employing a linear DNA, which is known as “Red-driven integration” (Proc. Natl. Acad. Sci. USA, 2000, 97 (12): 6640-6645, WO2005/010175), or by the method employing a plasmid containing a temperature-sensitive replication control region (Proc. Natl. Acad. Sci. USA, 2000, 97 (12): 6640-6645, U.S. Pat. Nos. 6,303,383 and 5,616,480). Furthermore, introduction of a site-specific mutation by gene replacement using homologous recombination as set forth above can also be performed by using a plasmid lacking the ability to replicate in the host.

Expression of the gene can also be attenuated by insertion of a transposon or an IS factor into the coding region of the gene (U.S. Pat. No. 5,175,107), or by conventional methods, such as mutagenesis treatment using UV irradiation or nitrosoguanidine (N-methyl-N′-nitro-N-nitrosoguanidine) treatment.

The presence of activity of the Nac protein can be detected by complementation of nac mutation by the method described, for example, (Muse, W. B. and Bender R. A., J. Bacteriol., 180, 5 1166-1173 (1998)). So, the reduced or absent activity of the Nac protein in the bacterium according the present invention can be determined when compared to the parent unmodified bacterium.

The presence or absence of the nac gene on the chromosome of a bacterium can be detected by well-known methods, including PCR, Southern blotting and the like. In addition, the level of gene expression can be estimated by measuring the amount of mRNA transcribed from the gene using various well-known methods, including Northern blotting, quantitative RT-PCR, and the like. Amount or molecular weight of the protein encoded by the gene can be measured by well-known methods, including SDS-PAGE followed by immunoblotting assay (Western blotting analysis), and the like.

Methods for preparation of plasmid DNA, digestion and ligation of DNA, transformation, selection of an oligonucleotide as a primer, and the like may be ordinary methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

L-Amino Acid Producing Bacteria

As a bacterium of the present invention, which is modified to attenuate expression of the nac gene, bacteria which are able to produce either an aromatic or a non-aromatic L-amino acid may be used.

The bacterium of the present invention can be obtained by attenuating expression of the nac gene in a bacterium which inherently has the ability to produce an L-amino acid. Alternatively, the bacterium of present invention can be obtained by imparting the ability to produce an L-amino acid to a bacterium already having attenuated expression of the nac gene.

L-Threonine-Producing Bacteria

Examples of parent strains for deriving the L-threonine-producing bacteria of the present invention include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli 472T23/pYN7 (ATCC 98081) (U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A), and the like.

The strain TDH-6 is deficient in the thrC gene, as well as being sucrose-assimilative, and the ilvA gene has a leaky mutation. This strain also has a mutation in the rhtA gene, which imparts resistance to high concentrations of threonine or homoserine. The strain B-3996 contains the plasmid pVIC40 which was obtained by inserting a thrA*BC operon which includes a mutant thrA gene into a RSF1010-derived vector. This mutant thrA gene encodes aspartokinase homoserine dehydrogenase I which is substantially desensitized to feedback inhibition by threonine. The strain B-3996 was deposited on Nov. 19, 1987 in the All-Union Scientific Center of Antibiotics (Nagatinskaya Street 3-A, 117105 Moscow, Russian Federation) under the accession number RIA 1867. The strain was also deposited in the Russian National Collection of Industrial Microorganisms (VKPM) (Russia, 117545 Moscow 1, Dorozhny proezd. 1) on Apr. 7, 1987 under the accession number B-3996.

E. coli VKPM B-5318 (EP 0593792B) may also be used as a parent strain for deriving L-threonine-producing bacteria of the present invention. The strain B-5318 is prototrophic with regard to isoleucine, and a temperature-sensitive lambda-phage C1 repressor and PR promoter replaces the regulatory region of the threonine operon in plasmid pVIC40. The strain VKPM B-5318 was deposited in the Russian National Collection of Industrial Microorganisms (VKPM) on May 3, 1990 under accession number of VKPM B-5318.

Preferably, the bacterium of the present invention is additionally modified to enhance expression of one or more of the following genes:
the mutant thrA gene which codes for aspartokinase homoserine dehydrogenase I resistant to feed back inhibition by threonine;
the thrB gene which codes for homoserine kinase;
the thrC gene which codes for threonine synthase;
the rhtA gene which codes for a putative transmembrane protein;
the asd gene which codes for aspartate-β-semialdehyde dehydrogenase; and
the aspC gene which codes for aspartate aminotransferase (aspartate transaminase);

The thrA gene which encodes aspartokinase homoserine dehydrogenase I of Escherichia coli has been elucidated (nucleotide positions 337 to 2799, GenBank accession NC_{—}000913.2, gi: 49175990). The thrA gene is located between the thrL and thrB genes on the chromosome of E. coli K-12. The thrB gene which encodes homoserine kinase of Escherichia coli has been elucidated (nucleotide positions 2801 to 3733, GenBank accession NC_{—}000913.2, gi: 49175990). The thrB gene is located between the thrA and thrC genes on the chromosome of E. coli K-12. The thrC gene which encodes threonine synthase of Escherichia coli has been elucidated (nucleotide positions 3734 to 5020, GenBank accession NC_{—}000913.2, gi: 49175990). The thrC gene is located between the thrB gene and the yaaX open reading frame on the chromosome of E. coli K-12. All three genes functions as a single threonine operon. To enhance expression of the threonine operon, the attenuator region which affects the transcription is desirably removed from the operon (WO2005/049808, WO2003/097839).

A mutant thrA gene which codes for aspartokinase homoserine dehydrogenase I resistant to feed back inhibition by threonine, as well as, the thrB and thrC genes can be obtained as one operon from well-known plasmid pVIC40 which is presented in the threonine producing E. coli strain VKPM B-3996. Plasmid pVIC40 is described in detail in U.S. Pat. No. 5,705,371.

The rhtA gene exists at 18 min on the E. coli chromosome close to the glnHPQ operon, which encodes components of the glutamine transport system. The rhtA gene is identical to ORF1 (ybiF gene, nucleotide positions 764 to 1651, GenBank accession number AAA218541, gi:440181) and located between the pexB and ompX genes. The unit expressing a protein encoded by the ORF1 has been designated the rhtA gene (rht: resistance to homoserine and threonine). Also, it was revealed that the rhtA23 mutation is an A-for-G substitution at position-1 with respect to the ATG start codon (ABSTRACTS of the 17th International Congress of Biochemistry and Molecular Biology in conjugation with Annual Meeting of the American Society for Biochemistry and Molecular Biology, San Francisco, Calif. Aug. 24-29, 1997, abstract No. 457, EP 1013765 A).

The asd gene of E. coli has already been elucidated (nucleotide positions 3572511 to 3571408, GenBank accession NC_{—}000913.1, gi:16131307), and can be obtained by PCR (polymerase chain reaction; refer to White, T. J. et al., Trends Genet., 5, 185 (1989)) utilizing primers prepared based on the nucleotide sequence of the gene. The asd genes of other microorganisms can be obtained in a similar manner.

Also, the aspC gene of E. coli has already been elucidated (nucleotide positions 983742 to 984932, GenBank accession NC_{—}000913.1, gi:16128895), and can be obtained by PCR. The aspC genes of other microorganisms can be obtained in a similar manner.

L-Lysine-Producing Bacteria

Examples of L-lysine-producing bacteria belonging to the genus Escherichia include mutants having resistance to an L-lysine analogue. The L-lysine analogue inhibits growth of bacteria belonging to the genus Escherichia, but this inhibition is fully or partially desensitized when L-lysine is present in the medium. Examples of the L-lysine analogue include, but are not limited to, oxalysine, lysine hydroxamate, S-(2-aminoethyl)-L-cysteine (AEC), γ-methyllysine, α-chlorocaprolactam and so forth. Mutants having resistance to these lysine analogues can be obtained by subjecting bacteria belonging to the genus Escherichia to a conventional artificial mutagenesis treatment. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, feedback inhibition of aspartokinase by L-lysine is desensitized.

The strain WC196 may be used as an L-lysine producing bacterium of Escherichia coli. This bacterial strain was bred by conferring AEC resistance to the strain W3110, which was derived from Escherichia coli K-12. The resulting strain was designated Escherichia coli AJ13069 strain and was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994 and received an accession number of FERM P-14690. Then, it was converted to an international deposit under the provisions of the Budapest Treaty on Sep. 29, 1995, and received an accession number of FERM BP-5252 (U.S. Pat. No. 5,827,698).

Examples of parent strains for deriving L-lysine-producing bacteria of the present invention also include strains in which expression of one or more genes encoding an L-lysine biosynthetic enzyme are enhanced. Examples of such genes include, but are not limited to, genes encoding dihydrodipicolinate synthase (dapA), aspartokinase (lysC), dihydrodipicolinate reductase (dapB), diaminopimelate decarboxylase (lysA), diaminopimelate dehydrogenase (ddh) (U.S. Pat. No. 6,040,160), phosphoenolpyrvate carboxylase (ppc), aspartate semialdehyde dehydrogenease (asd), and aspartase (aspA) (EP 1253195 A). In addition, the parent strains may have an increased level of expression of the gene involved in energy efficiency (cyo) (EP 1170376 A), the gene encoding nicotinamide nucleotide transhydrogenase (pntAB) (U.S. Pat. No. 5,830,716), the ybjE gene (WO2005/073390), or combinations thereof.

Examples of parent strains for deriving L-lysine-producing bacteria of the present invention also include strains having decreased or eliminated activity of an enzyme that catalyzes a reaction for generating a compound other than L-lysine by branching off from the biosynthetic pathway of L-lysine. Examples of the enzymes that catalyze a reaction for generating a compound other than L-lysine by branching off from the biosynthetic pathway of L-lysine include homoserine dehydrogenase, lysine decarboxylase (U.S. Pat. No. 5,827,698), and the malic enzyme (WO2005/010175).

L-Cysteine-Producing Bacteria

Examples of parent strains for deriving L-cysteine-producing bacteria of the present invention include, but are not limited to, strains belonging to the genus Escherichia, such as E. coli JM15 which is transformed with different cysE alleles coding for feedback-resistant serine acetyltransferases (U.S. Pat. No. 6,218,168, Russian patent application 2003121601); E. coli W3110 having over-expressed genes which encode proteins suitable for secreting substances toxic for cells (U.S. Pat. No. 5,972,663); E. coli strains having lowered cysteine desulfohydrase activity (JP11155571A2); E. coli W3110 with increased activity of a positive transcriptional regulator for cysteine regulon encoded by the cysB gene (WO0127307A1), and the like.

L-Leucine-Producing Bacteria

Examples of parent strains for deriving L-leucine-producing bacteria of the present invention include, but are not limited to, strains belonging to the genus Escherichia, such as E. coli strains resistant to leucine (for example, the strain 57 (VKPM B-7386, U.S. Pat. No. 6,124,121)) or leucine analogs including β-2-thienylalanine, 3-hydroxyleucine, 4-azaleucine, 5,5,5-trifluoroleucine (JP 62-34397 B and JP 8-70879 A); E. coli strains obtained by the gene engineering method described in WO96/06926; E. coli H-9068 (JP 8-70879 A), and the like.

The bacterium of the present invention may be improved by enhancing the expression of one or more genes involved in L-leucine biosynthesis. Examples include genes of the leuABCD operon, which are preferably represented by a mutant leuA gene coding for isopropylmalate synthase not subject to feedback inhibition by L-leucine (U.S. Pat. 6,403,342). In addition, the bacterium of the present invention may be improved by enhancing the expression of one or more genes coding for proteins which excrete L-amino acid from the bacterial cell. Examples of such genes include the b2682 and b2683 genes (ygaZH genes) (EP 1239041 A2).

L-Histidine-Producing Bacteria

Examples of parent strains for deriving L-histidine-producing bacteria of the present invention include, but are not limited to, strains belonging to the genus Escherichia, such as E. coli strain 24 (VKPM B-5945, RU2003677); E. coli strain 80 (VKPM B-7270, RU2119536); E. coli NRRL B-12116-B12121 (U.S. Pat. No. 4,388,405); E. coli H-9342 (FERM BP-6675) and H-9343 (FERM BP-6676) (U.S. Pat. No. 6,344,347); E. coli H-9341 (FERM BP-6674) (EP1085087); E. coli A180/pFM201 (U.S. Pat. No. 6,258,554) and the like.

Examples of parent strains for deriving L-histidine-producing bacteria of the present invention also include strains in which expression of one or more genes encoding an L-histidine biosynthetic enzyme are enhanced. Examples of such genes include genes encoding ATP phosphoribosyltransferase (hisG), phosphoribosyl AMP cyclohydrolase (hisI), phosphoribosyl-ATP pyrophosphohydrolase (hisIE), phosphoribosylformimino-5-aminoimidazole carboxamide ribotide isomerase (hisA), amidotransferase (hisH), histidinol phosphate aminotransferase (hisC), histidinol phosphatase (hisB), histidinol dehydrogenase (hisD), and so forth.

It is known that the L-histidine biosynthetic enzymes encoded by hisG and hisBHAFI are inhibited by L-histidine, and therefore an L-histidine-producing ability can also be efficiently enhanced by introducing a mutation conferring resistance to the feedback inhibition into ATP phosphoribosyltransferase (Russian Patent Nos. 2003677 and 2119536).

Specific examples of strains having an L-histidine-producing ability include E. coli FERM-P 5038 and 5048 which have been introduced with a vector carrying a DNA encoding an L-histidine-biosynthetic enzyme (JP 56-005099 A), E. coli strains introduced with rht, a gene for an amino acid-export (EP1016710A), E. coli 80 strain imparted with sulfaguanidine, DL-1,2,4-triazole-3-alanine, and streptomycin-resistance (VKPM B-7270, Russian Patent No. 2119536), and so forth.

L-Glutamic Acid-Producing Bacteria

Examples of parent strains for deriving L-glutamic acid-producing bacteria of the present invention include, but are not limited to, strains belonging to the genus Escherichia, such as E. coli VL334thrC⁺ (EP 1172433). E. coli VL334 (VKPM B-1641) is an L-isoleucine and L-threonine auxotrophic strain having mutations in thrC and ilvA genes (U.S. Pat. No. 4,278,765). A wild-type allele of the thrC gene was transferred by the method of general transduction using a bacteriophage P1 grown on the wild-type E. coli strain K12 (VKPM B-7) cells. As a result, an L-isoleucine auxotrophic strain VL334thrC⁺ (VKPM B-8961), which is able to produce L-glutamic acid, was obtained.

Examples of parent strains for deriving the L-glutamic acid-producing bacteria of the present invention include, but are not limited to, strains in which expression of one or more genes encoding an L-glutamic acid biosynthetic enzyme are enhanced. Examples of such genes include genes encoding glutamate dehydrogenase (gdhA), glutamine synthetase (glnA), glutamate synthetase (gltAB), isocitrate dehydrogenase (icdA), aconitate hydratase (acnA, acnB), citrate synthase (gltA), phosphoenolpyruvate carboxylase (ppc), pyruvate carboxylase (pyc), pyruvate dehydrogenase (aceEF, lpdA), pyruvate kinase (pykA, pykF), phosphoenolpyruvate synthase (ppsA), enolase (eno), phosphoglyceromutase (pgmA, pgmI), phosphoglycerate kinase (pgk), glyceraldehyde-3-phophate dehydrogenase (gapA), triose phosphate isomerase (tpiA), fructose bisphosphate aldolase (fbp), phosphofructokinase (pfkA, pfkB), and glucose phosphate isomerase (pgi).

Examples of strains modified so that expression of the citrate synthetase gene, the phosphoenolpyruvate carboxylase gene, and/or the glutamate dehydrogenase gene is/are enhanced include those disclosed in EP1078989A, EP955368A, and EP952221A.

Examples of parent strains for deriving the L-glutamic acid-producing bacteria of the present invention also include strains having decreased or eliminated activity of an enzyme that catalyzes synthesis of a compound other than L-glutamic acid by branching off from an L-glutamic acid biosynthesis pathway. Examples of such enzymes include isocitrate lyase (aceA), α-ketoglutarate dehydrogenase (sucA), phosphotransacetylase (pta), acetate kinase (ack), acetohydroxy acid synthase (ilvG), acetolactate synthase (ilvI), formate acetyltransferase (pfl), lactate dehydrogenase (ldh), and glutamate decarboxylase (gadAB). Bacteria belonging to the genus Escherichia deficient in the α-ketoglutarate dehydrogenase activity or having a reduced α-ketoglutarate dehydrogenase activity and methods for obtaining them are described in U.S. Pat. Nos. 5,378,616 and 5,573,945. Specifically, these strains include the following:

E. coli W3110sucA:: Kmr

E. coli AJ12624 (FERM BP-3853)

E. coli AJ12628 (FERM BP-3854)

E. coli AJ12949 (FERM BP-4881)

E. coli W3110sucA::Kmr is a strain obtained by disrupting the α-ketoglutarate dehydrogenase gene (hereinafter referred to as “sucA gene”) of E. coli W3110. This strain is completely deficient in the α-ketoglutarate dehydrogenase.

Other examples of L-glutamic acid-producing bacterium include those which belong to the genus Escherichia and have resistance to an aspartic acid antimetabolite. These strains can also be deficient in the α-ketoglutarate dehydrogenase activity and include, for example, E. coli AJ13199 (FERM BP-5807) (U.S. Pat. No. 5,908,768), FFRM P-12379, which additionally has a low L-glutamic acid decomposing ability (U.S. Pat. No. 5,393,671); AJ13138 (FERM BP-5565) (U.S. Pat. No. 6,110,714), and the like.

Examples of L-glutamic acid-producing bacteria, include mutant strains belonging to the genus Pantoea which are deficient in the α-ketoglutarate dehydrogenase activity or have a decreased α-ketoglutarate dehydrogenase activity, and can be obtained as described above. Such strains include Pantoea ananatis AJ13356. (U.S. Pat. No. 6,331,419). Pantoea ananatis AJ13356 was deposited at the National Institute of Bioscience and Human-Technology, Agency of Industrial Science and Technology, Ministry of International Trade and Industry (currently, National Institute of Advanced Industrial Science and Technology, International Patent Organism Depositary, Central 6, 1-1, Higashi 1-Chome, Tsukuba-shi, Ibaraki-ken, 305-8566, Japan) on Feb. 19, 1998 under an accession number of FERM P-16645. It was then converted to an international deposit under the provisions of Budapest Treaty on Jan. 11, 1999 and received an accession number of FERM BP-6615. Pantoea ananatis AJ13356 is deficient in the α-ketoglutarate dehydrogenase activity as a result of disruption of the αAKGDH-E1 subunit gene (sucA). The above strain was identified as Enterobacter agglomerans when it was isolated and deposited as the Enterobacter agglomerans AJ13356. However, it was recently re-classified as Pantoea ananatis on the basis of nucleotide sequencing of 16S rRNA and so forth. Although AJ13356 was deposited at the aforementioned depository as Enterobacter agglomerans, for the purposes of this specification, they are described as Pantoea ananatis.

L-Phenylalanine-Producing Bacteria

Examples of parent strains for deriving L-phenylalanine-producing bacteria of the present invention include, but are not limited to, strains belonging to the genus Escherichia, such as E. coli AJ12739 (tyrA::Tn10, tyrR) (VKPM B-8197); E. coli HW1089 (ATCC 55371) harboring the mutant pheA34 gene (U.S. Pat. No. 5,354,672); E. coli MWEC101-b (KR8903681); E. coli NRRL B-12141, NRRL B-12145, NRRL B-12146 and NRRL B-12147 (U.S. Pat. No. 4,407,952). Also, as a parent strain, E. coli K-12 [W3110 (tyrA)/pPHAB (FERM BP-3566), E. coli K-12 [W3110 (tyrA)/pPHAD] (FERM BP-12659), E. coli K-12 [W3110 (tyrA)/pPHATerm] (FERM BP-12662) and E. coli K-12 [W3110 (tyrA)/pBR-aroG4, pACMAB] named as AJ 12604 (FERM BP-3579) may be used (EP 488424 B1). Furthermore, L-phenylalanine producing bacteria belonging to the genus Escherichia with an enhanced activity of the protein encoded by the yedA gene or the yddG gene may also be used (U.S. patent applications 2003/0148473 A1 and 2003/0157667 A1).

L-Tryptophan-Producing Bacteria

Examples of parent strains for deriving the L-tryptophan-producing bacteria of the present invention include, but are not limited to, strains belonging to the genus Escherichia, such as E. coli JP4735/pMU3028 (DSM10122) and JP6015/pMU91 (DSM10123) deficient in the tryptophanyl-tRNA synthetase encoded by mutant trpS gene (U.S. Pat. No. 5,756,345); E. coli SV164 (pGH5) having a serA allele encoding phosphoglycerate dehydrogenase not subject to feedback inhibition by serine and a trpE allele encoding anthranilate synthase not subject to feedback inhibition by tryptophan (U.S. Pat. No. 6,180,373); E. coli AGX17 (pGX44) (NRRL B-12263) and AGX6(pGX50)aroP (NRRL B-12264) deficient in the enzyme tryptophanase (U.S. Pat. No. 4,371,614); E. coli AGX17/pGX50,pACKG4-pps in which a phosphoenolpyruvate-producing ability is enhanced (WO9708333, U.S. Pat. No. 6,319,696), and the like may be used. L-tryptophan-producing bacteria belonging to the genus Escherichia with an enhanced activity of the protein encoded by the yedA gene or the yddG gene may also be used (U.S. patent applications 2003/0148473 A1 and 2003/0157667 A1).

Examples of parent strains for deriving the L-tryptophan-producing bacteria of the present invention also include strains in which one or more activities of the enzymes selected from anthranilate synthase, phosphoglycerate dehydrogenase, and tryptophan synthase are enhanced. The anthranilate synthase and phosphoglycerate dehydrogenase are both subject to feedback inhibition by L-tryptophan and L-serine, so that a mutation desensitizing the feedback inhibition may be introduced into these enzymes. Specific examples of strains having such a mutation include a E. coli SV164 which harbors desensitized anthranilate synthase and a transformant strain obtained by introducing into the E. coli SV164 the plasmid pGH5 (WO 94/08031), which contains a mutant serA gene encoding feedback-desensitized phosphoglycerate dehydrogenase.

Examples of parent strains for deriving the L-tryptophan-producing bacteria of the present invention also include strains into which the tryptophan operon which contains a gene encoding desensitized anthranilate synthase has been introduced (JP 57-71397 A, JP 62-244382 A, U.S. Pat. No. 4,371,614). Moreover, L-tryptophan-producing ability may be imparted by enhancing expression of a gene which encodes tryptophan synthase, among tryptophan operons (trpBA). The tryptophan synthase consists of α and β subunits which are encoded by the trpA and trpB genes, respectively. In addition, L-tryptophan-producing ability may be improved by enhancing expression of the isocitrate lyase-malate synthase operon (WO2005/103275).

L-Proline-Producing Bacteria

Examples of parent strains for deriving L-proline-producing bacteria of the present invention include, but are not limited to, strains belonging to the genus Escherichia, such as E. coli 702ilvA (VKPM B-8012) which is deficient in the ilvA gene and is able to produce L-proline (EP 1172433). The bacterium of the present invention may be improved by enhancing the expression of one or more genes involved in L-proline biosynthesis. Examples of such genes for L-proline producing bacteria which are preferred include the proB gene coding for glutamate kinase of which feedback inhibition by L-proline is desensitized (DE Patent 3127361). In addition, the bacterium of the present invention may be improved by enhancing the expression of one or more genes coding for proteins excreting L-amino acid from bacterial cell. Such genes are exemplified by b2682 and b2683 genes (ygaZH genes) (EP1239041 A2).

Examples of bacteria belonging to the genus Escherichia, which have an activity to produce L-proline include the following E. coli strains: NRRL B-12403 and NRRL B-12404 (GB Patent 2075056), VKPM B-8012 (Russian patent application 2000124295), plasmid mutants described in DE Patent 3127361, plasmid mutants described by Bloom F. R. et al (The 15th Miami winter symposium, 1983, p.34), and the like.

L-Arginine-Producing Bacteria

Examples of parent strains for deriving L-arginine-producing bacteria of the present invention include, but are not limited to, strains belonging to the genus Escherichia, such as E. coli strain 237 (VKPM B-7925) (U.S. Patent Application 2002/058315 A1) and its derivative strains harboring mutant N-acetylglutamate synthase (Russian Patent Application No. 2001112869), E. coli strain 382 (VKPM B-7926) (EP1170358A1), an arginine-producing strain into which argA gene encoding N-acetylglutamate synthetase is introduced therein (EP1170361A1), and the like.

Examples of parent strains for deriving L-arginine producing bacteria of the present invention also include strains in which expression of one or more genes encoding an L-arginine biosynthetic enzyme are enhanced. Examples of such genes include genes encoding N-acetylglutamyl phosphate reductase (argC), ornithine acetyl transferase (argJ), N-acetylglutamate kinase (argB), acetylornithine transaminase (argD), ornithine carbamoyl transferase (argF), argininosuccinic acid synthetase (argG), argininosuccinic acid lyase (argH), and carbamoyl phosphate synthetase (carAB).

L-Valine-Producing Bacteria

Examples of parent strains for deriving L-valine-producing bacteria of the present invention include, but are not limited to, strains which have been modified to overexpress the ilvGMEDA operon (U.S. Pat. No. 5,998,178). It is desirable to remove the region of the ilvGMEDA operon which is required for attenuation so that expression of the operon is not attenuated by L-valine that is produced. Furthermore, the ilvA gene in the operon is desirably disrupted so that threonine deaminase activity is decreased.

Examples of parent strains for deriving L-valine-producing bacteria of the present invention include also include mutants having a mutation of amino-acyl t-RNA synthetase (U.S. Pat. No. 5,658,766). For example, E. coli VL1970, which has a mutation in the ileS gene encoding isoleucine tRNA synthetase, can be used. E. coli VL1970 has been deposited in the Russian National Collection of Industrial Microorganisms (VKPM) (Russia, 113545 Moscow, 1 Dorozhny Proezd, 1) on Jun. 24, 1988 under accession number VKPM B-4411.

Furthermore, mutants requiring lipoic acid for growth and/or lacking H⁺-ATPase can also be used as parent strains (WO96/06926).

L-Isoleucine-Producing Bacteria

Examples of parent strains for deriving L-isoleucine producing bacteria of the present invention include, but are not limited to, mutants having resistance to 6-dimethylaminopurine (JP 5-304969 A), mutants having resistance to an isoleucine analogue such as thiaisoleucine and isoleucine hydroxamate, and mutants additionally having resistance to DL-ethionine and/or arginine hydroxamate (JP 5-130882 A). In addition, recombinant strains transformed with genes encoding proteins involved in L-isoleucine biosynthesis, such as threonine deaminase and acetohydroxate synthase, can also be used as parent strains (JP 2-458 A, FR 0356739, and U.S. Pat. No. 5,998,178).

2. Method of the Present Invention

The method of the present invention is a method for producing an L-amino acid by cultivating the bacterium of the present invention in a culture medium to produce and excrete the L-amino acid into the medium, and collecting the L-amino acid from the medium.

In the present invention, the cultivation, collection, and purification of an L-amino acid from the medium and the like may be performed in a manner similar to conventional fermentation methods wherein an amino acid is produced using a bacterium.

The medium used for the culture may be either a synthetic or natural medium, so long as the medium includes a carbon source and a nitrogen source and minerals and, if necessary, appropriate amounts of nutrients which the bacterium requires for growth. The carbon source may include various carbohydrates such as glucose and sucrose, and various organic acids. Depending on the mode of assimilation of the used microorganism, alcohol, including ethanol and glycerol, may be used. As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used. As vitamins, thiamine, yeast extract, and the like, can be used.

The cultivation is preferably performed under aerobic conditions, such as a shaking culture, and a stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to accumulation of the target L-amino acid in the liquid medium.

After cultivation, solids such as cells can be removed from the liquid medium by centrifugation or membrane filtration, and then the L-amino acid can be collected and purified by ion-exchange, concentration, and/or crystallization methods.

### EXAMPLES

The present invention will be more concretely explained below with reference to the following non-limiting Examples.

### Example 1

### Construction of a Strain With an Inactivated Nac Gene

1. Deletion of the Nac Gene.

A strain in which the nac gene is deleted was constructed by the method initially developed by Datsenko, K. A. and Wanner, B. L. (Proc. Natl. Acad. Sci. USA, 2000, 97(12), 6640-6645) called “Red-driven integration”. According to this procedure, the PCR primers nac L (SEQ ID NO: 3) and nac R (SEQ ID NO: 4), which are homologous to both the regions adjacent to the nac gene and the gene conferring antibiotic resistance, respectively, in the template plasmid, were constructed. The plasmid pACYC184 (NBL Gene Sciences Ltd., UK) (GenBank/EMBL accession number X06403) was used as a template in the PCR reaction. Conditions for PCR were as follows: denaturation step: 3 min at 95° C.; profile for two first cycles: 1 min at 95° C., 30 sec at 50° C., 40 sec at 72° C.; profile for the last 25 cycles: 30 sec at 95° C., 30 sec at 54° C., 40 sec at 72° C.; final step: 5 min at 72° C.

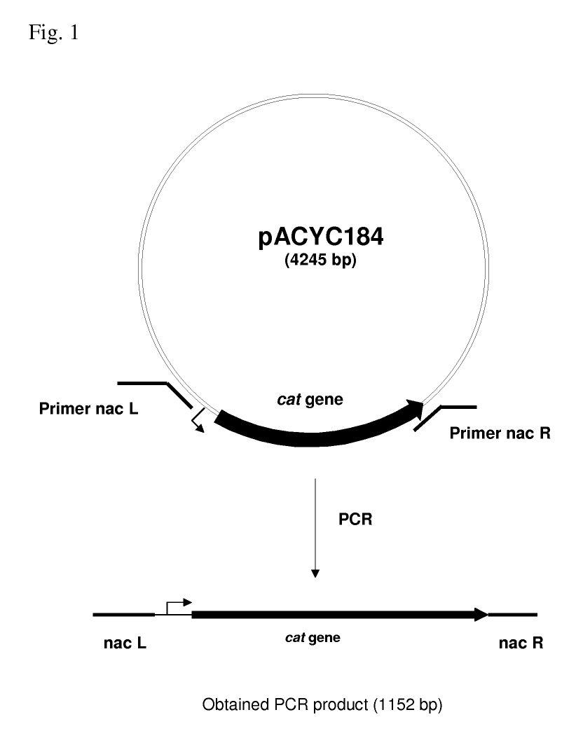

A 1152 bp PCR product (FIG. 1) was obtained and purified in agarose gel and was used for electroporation of E. coli MG1655 (ATCC 700926), which contains the plasmid pKD46 having a temperature-sensitive replication. The plasmid pKD46 (Datsenko, K. A. and Wanner, B. L., Proc. Natl. Acad. Sci. USA, 2000, 97:12:6640-45) includes a 2,154 nucleotide (31088-33241) DNA fragment of phage λ (GenBank accession No. J02459), and contains genes of the λ Red homologous recombination system (γ, β, exo genes) under the control of the arabinose-inducible P_{araB} promoter. The plasmid pKD46 is necessary for integration of the PCR product into the chromosome of strain MG1655.

Electrocompetent cells were prepared as follows: E. coli MG1655/pKD46 was grown overnight at 30° C. in LB medium containing ampicillin (100 mg/l), and the culture was diluted 100 times with 5 ml of SOB medium (Sambrook et al, “Molecular Cloning A Laboratory Manual, Second Edition”, Cold Spring Harbor Laboratory Press (1989)) containing ampicillin and L-arabinose (1 mM). The cells were grown with aeration at 30° C. to an OD₆₀₀ of ≈0.6 and then were made electrocompetent by concentrating 100-fold and washing three times with ice-cold deionized H₂O. Electroporation was performed using 70 μl of cells and ≈100 ng of PCR product. Cells after electroporation were incubated with 1 ml of SOC medium (Sambrook et al, “Molecular Cloning A Laboratory Manual, Second Edition”, Cold Spring Harbor Laboratory Press (1989)) at 37° C. for 2.5 hours and then were plated onto L-agar containing chloramphenicol (30 μg/ml) and grown at 37° C. to select Cm^{R} recombinants. Then, to eliminate the pKD46 plasmid, 2 passages on L-agar with Cm at 42° C. were performed and the obtained colonies were tested for sensitivity to ampicillin.

2. Verification of the Nac Gene Deletion by PCR.

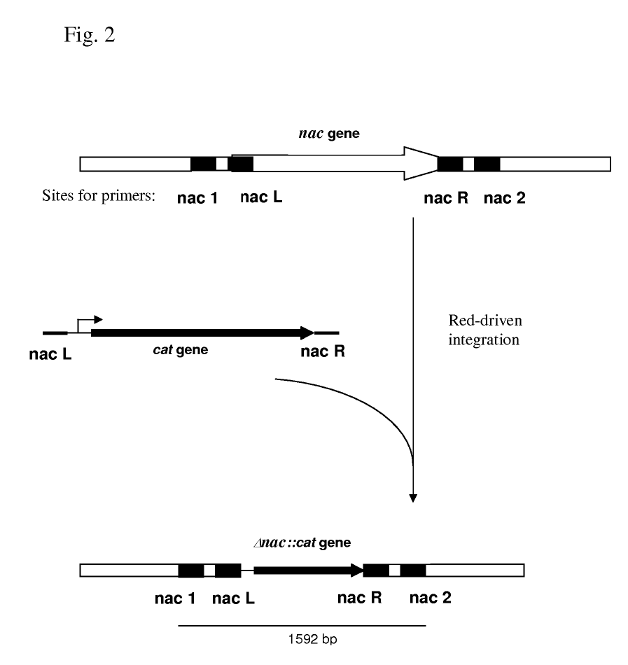

The mutants, which have the nac gene deleted, and marked with the Cm resistance gene, were verified by PCR. Locus-specific primers nac 1 (SEQ ID NO: 5) and nac 2 (SEQ ID NO: 6) were used in PCR for verification. Conditions for PCR verification were as follows: denaturation step: 3 min at 94° C.; profile for the 30 cycles: 30 sec at 94° C., 30 sec at 54° C., 1 min at 72° C.; final step: 7 min at 72° C. The PCR product obtained in the reaction with the cells of the parental nac⁺ strain MG1655 as the template was 1350 bp in length. The PCR product obtained in the reaction with the cells of the mutant strain as the template was 1592 bp in length (FIG. 2). The mutant strain was named MG1655 Δnac::cat.

### Example 2

### Production of L-Threonine by E. Coli B-3996-ΔNac

To test the effect of inactivation of the nac gene on threonine production, DNA fragments from the chromosome of the above-described E. coli MG1655 Δnac::cat were transferred to the threonine-producing E. coli strain VKPM B-3996 by P1 transduction (Miller, J. H. (1972) Experiments in Molecular Genetics, Cold Spring Harbor Lab. Press, Plainview, N.Y.) to obtain the strain B-3996-Δnac.

Both E. coli B-3996 and B-3996-Δnac were grown for 18-24 hours at 37° C. on L-agar plates. To obtain a seed culture, the strains were grown on a rotary shaker (250 rpm) at 32° C. for 18 hours in 20×200 mm test tubes containing 2 ml of L-broth with 4% sucrose. Then, the fermentation medium was inoculated with 0.21 ml (10%) seed material. The fermentation was performed in 2 ml of minimal medium for fermentation in 20×200 mm test tubes. Cells were grown for 72 hours at 32° C. with shaking at 250 rpm.

After cultivation, the amount of L-threonine which had accumulated in the medium was determined by paper chromatography using the following mobile phase:butanol:acetic acid:water=4:1:1 (v/v). A solution (2%) of ninhydrin in acetone was used as a visualizing reagent. A spot containing L-threonine was cut out, L-threonine was eluted in 0.5% water solution of CdCl₂, and the amount of L-threonine was estimated spectrophotometrically at 540 nm. The results of 8 independent test tube fermentations are shown in Table 1.

The composition of the fermentation medium (g/l) was as follows:

| | |
|---|---|
| Glucose | 80.0 |
| (NH₄)₂SO₄ | 22.0 |
| NaCl | 0.8 |
| KH₂PO₄ | 2.0 |
| MgSO₄•7H₂O | 0.8 |
| FeSO₄•7H₂O | 0.02 |
| MnSO₄•5H₂O | 0.02 |
| Thiamine HCl | 0.0002 |
| Yeast extract | 1.0 |
| CaCO₃ | 30.0 |

Glucose and magnesium sulfate were sterilized separately. CaCO₃ was sterilized by dry-heat at 180° C. for 2 hours. The pH was adjusted to 7.0. Antibiotic was introduced into the medium after sterilization.

TABLE 1

| Strain | OD₅₄₀ | Amount of L-threonine, g/l |
|---|---|---|
| B-3996 | 25.2 ± 2.6 | 28.4 ± 0.7 |
| B-3996-Δnac | 27.7 ± 4.1 | 31.9 ± 1.0 |

It can be seen from Table 1 that B-3996-Δnac was able to produce a higher amount of L-threonine as compared with B-3996.

### Example 3

### Production of L-Lysine by E. Coli WC196-ΔNac

To test the effect of inactivation of the nac gene on lysine production, DNA fragments from the chromosome of the above-described E. coli MG1655 Δnac::cat were transferred to the lysine-producing E. coli strain WC196 (FERM BP-5252) by P1 transduction (Miller, J. H. (1972) Experiments in Molecular Genetics, Cold Spring Harbor Lab. Press, Plainview, N.Y.) to obtain the strain WC196-Δnac.

To obtain a seed culture, both E. coli WC196 and WC196-Δnac were grown on a rotary shaker (250 rpm) at 32° C. for 18 hours in 20×200-mm test tubes containing 2 ml of medium diluted two times compared to the fermentation medium described below. Then 0.21 ml (10%) of the seed culture was inoculated into 2 ml of the fermentation medium in 20×200 mm test tubes. The fermentation was performed at 32° C. for 24 hours with shaking at 250 rpm.

After cultivation, the amount of L-lysine which had accumulated in the medium was determined by paper chromatography using the following mobile phase: butanol-acetic acid-water=4:1:1 (v/v). A solution of ninhydrin (2%) in acetone was used as a visualizing reagent. A spot containing L-lysine was cut out, L-lysine was eluted with 0.5% water solution of CdCl₂, and the amount of L-lysine was estimated spectrophotometrically at 540 nm. The results of five independent test-tube fermentations are shown in Table 2.

The composition of the fermentation medium (g/l) was as follows:

| | |
|---|---|
| Glucose | 40.0 |
| (NH₄)₂SO₄ | 24.0 |
| KH₂PO₄ | 1.0 |
| MgSO₄•7H₂O | 1.0 |
| FeSO₄•7H₂O | 0.01 |
| MnSO₄•5H₂O | 0.01 |
| Yeast extract | 2.0 |
| CaCO₃ | 30.0 |

Glucose, potassium phosphate and magnesium sulfate were sterilized separately. CaCO₃ was sterilized by dry-heat at 180° C. for 2 hours. The pH was adjusted to 7.0.

TABLE 2

| Strain | OD₅₄₀ | Amount of L-lysine, g/l |
|---|---|---|
| WC196 | 24.8 ± 0.4 | 1.9 ± 0.1 |
| WC196-Δnac | 18.3 ± 0.3 | 2.3 ± 0.2 |

As follows from Table 2, WC196-Δnac was able to produce a higher amount of L-lysine, as compared with WC196.

### Example 4

### Production of L-Cysteine by E. Coli JM15(ydeD)-ΔNac

To test the effect of inactivation of the nac gene on L-cysteine production, DNA fragments from the chromosome of the above-described E. coli MG1655 Δnac::cat can be transferred to the E. coli L-cysteine producing strain JM15(ydeD) by P1 transduction (Miller, J. H. (1972) Experiments in Molecular Genetics, Cold Spring Harbor Lab. Press, Plainview, N.Y.) to obtain the strain JM15(ydeD)-Δnac.

E. coli JM15(ydeD) is a derivative of E. coli JM15 (U.S. Pat. No. 6,218,168) which can be transformed with DNA having the ydeD gene, which codes for a membrane protein, and is not involved in a biosynthetic pathway of any L-amino acid (U.S. Pat. No. 5,972,663). The strain JM15 (CGSC# 5042) can be obtained from The Coli Genetic Stock Collection at the E. coli Genetic Resource Center, MCD Biology Department, Yale University

(http://cgsc.biology.yale.edu/).

Fermentation conditions for evaluation of L-cysteine production are described in detail in Example 6 of U.S. Pat. No. 6,218,168.

### Example 5

### Production of L-Leucine by E. Coli 57-ΔNac

To test the effect of inactivation of the nac gene on L-leucine production, DNA fragments from the chromosome of the above-described E. coli MG1655 Δnac::cat can be transferred to the E. coli L-leucine producing strain 57 (VKPM B-7386, U.S. Pat. No. 6,124,121) by P1 transduction (Miller, J. H. (1972) Experiments in Molecular Genetics, Cold Spring Harbor Lab. Press, Plainview, N.Y.) to obtain the strain 57-Δnac. The strain 57 has been deposited in the Russian National Collection of Industrial Microorganisms (VKPM) (Russia, 117545 Moscow, 1 Dorozhny proezd, 1) on May 19, 1997 under accession number VKPM B-7386.

Both E. coli 57 and 57-Δnac can be cultured for 18-24 hours at 37° C. on L-agar plates. To obtain a seed culture, the strains can be grown on a rotary shaker (250 rpm) at 32° C. for 18 hours in 20×200 mm test tubes containing 2 ml of L-broth with 4% sucrose. Then, the fermentation medium can be inoculated with 0.21 ml (10%) seed material. The fermentation can be performed in 2 ml of minimal medium for fermentation in 20×200 mm test tubes. Cells can be grown for 48-72 hours at 32° C. with shaking at 250 rpm. The amount of L-leucine can be measured by paper chromatography (liquid phase composition: butanol-acetic acid-water=4:1:1)

The composition of the fermentation medium (g/l) is as follows (pH 7.2):

| | |
|---|---|
| Glucose | 60.0 |
| (NH₄)₂SO₄ | 25.0 |
| K₂HPO₄ | 2.0 |
| MgSO₄•7H₂O | 1.0 |
| Thiamine | 0.01 |
| CaCO₃ | 25.0 |

Glucose and CaCO₃ are sterilized separately.

### Example 6

### Production of L-Histidine by E. Coli 80-ΔNac

To test the effect of inactivation of the nac gene on L-histidine production, DNA fragments from the chromosome of the above-described E. coli MG1655 Δnac::cat can be transferred to the histidine-producing E. coli strain 80 by P1 transduction (Miller, J. H. (1972) Experiments in Molecular Genetics, Cold Spring Harbor Lab. Press, Plainview, N.Y.) to obtain the strain 80-Δnac. The strain 80 has been described in Russian patent 2119536 and deposited in the Russian National Collection of Industrial Microorganisms (Russia, 117545 Moscow, 1 Dorozhny proezd, 1) on Oct. 15, 1999 under accession number VRPM B-7270 and then converted to a deposit under the Budapest Treaty on Jul. 12, 2004.

Both E. coli 80 and 80-Δnac can be cultivated in L-broth for 6 hours at 29° C. Then, 0.1 ml of the cultures can each be inoculated into 2 ml of fermentation medium in 20×200 mm test tube and cultivated for 65 hours at 29° C. with a rotary shaker (350 rpm). After cultivation, the amount of histidine, which accumulates in the medium, can be determined by paper chromatography. The paper can be developed with a mobile phase: n-butanol: acetic acid:water=4:1:1 (v/v). A solution of ninhydrin (0.5%) in acetone can be used as a visualizing reagent.

The composition of the fermentation medium (g/l) is as follows (pH 6.0):

| | |
|---|---|
| Glucose | 100.0 |
| Mameno (soybean hydrolysate) | 0.2 of as total nitrogen |
| L-proline | 1.0 |
| (NH₄)₂SO₄ | 25.0 |
| KH₂PO₄ | 2.0 |
| MgSO₄•7H₂O | 1.0 |
| FeSO₄•7H₂O | 0.01 |
| MnSO₄ | 0.01 |
| Thiamine | 0.001 |
| Betaine | 2.0 |
| CaCO₃ | 60.0 |

Glucose, proline, betaine and CaCO₃ are sterilized separately. pH is adjusted to 6.0 before sterilization.

### Example 7

### Production of L-Glutamate by E. Coli VL334thrC⁺-ΔNac

To test the effect of inactivation of the nac gene on L-glutamate production, DNA fragments from the chromosome of the above-described E. coli MG1655 Δnac::cat can be transferred to the E. coli L-glutamate producing strain VL334thrC⁺ (EP 1172433) by P1 transduction (Miller, J. H. (1972) Experiments in Molecular Genetics, Cold Spring Harbor Lab. Press, Plainview, N.Y.) to obtain the strain VL334thrC⁺-Δnac. The strain L334thrC⁺ has been deposited in the Russian National Collection of Industrial Microorganisms (VKPM) (Russia, 117545 Moscow, 1 Dorozhny proezd, 1) on Dec. 6, 2004 under the accession number B-8961 and then converted to a deposit under the Budapest Treaty on Dec. 8, 2004.

Both strains, VL334thrC⁺ and VL334thrC⁺-Δnac, can be grown for 18-24 hours at 37° C. on L-agar plates. Then, one loop of the cells can be transferred into test tubes containing 2 ml of fermentation medium. The fermentation medium contains 60g/l glucose, 25 g/l ammonium sulfate, 2 g/l KH₂PO₄, 1 g/l MgSO₄, 0.1 mg/ml thiamine, 70 μg/ml L-isoleucine and 25 g/l CaCO₃ (pH 7.2). Glucose and CaCO₃ are sterilized separately. Cultivation can be carried out at 30° C. for 3 days with shaking. After the cultivation, the amount of L-glutamic acid produced can be determined by paper chromatography (liquid phase composition: butanol-acetic acid-water=4:1:1) with subsequent staining by ninhydrin (1% solution in acetone) and further elution of the compounds in 50% ethanol with 0.5% CdCl₂.

### Example 8

### Production of L-Phenylalanine by E. Coli AJ12739-ΔNac

To test the effect of inactivation of the nac gene on L-phenylalanine production, DNA fragments from the chromosome of the above-described E. coli MG1655 Δnac::cat can be transferred to the phenylalanine-producing E. coli strain AJ12739 by P1 transduction (Miller, J. H. (1972) Experiments in Molecular Genetics, Cold Spring Harbor Lab. Press, Plainview, N.Y.) to obtain the strain AJ12739-Δnac. The strain AJ12739 has been deposited in the Russian National Collection of Industrial Microorganisms (VKPM) (Russia, 117545 Moscow, 1 Dorozhny proezd, 1) on Nov. 6, 2001 under accession number VKPM B-8197 and then converted to a deposit under the Budapest Treaty on Aug. 23, 2002.

Both strains, AJ12739-Δnac and AJ12739, can be cultivated at 37° C. for 18 hours in a nutrient broth. 0.3 ml of the cultures can each be inoculated into 3 ml of a fermentation medium in a 20×200 mm test tube and cultivated at 37° C. for 48 hours with a rotary shaker. After cultivation, the amount of phenylalanine, which accumulates in the medium can be determined by TLC. 10×15 cm TLC plates coated with 0.11 mm layers of Sorbfil silica gel without fluorescent indicator (Stock Company Sorbpolymer, Krasnodar, Russia) can be used. Sorbfil plates can be developed with a mobile phase: propan-2-ol: ethylacetate: 25% aqueous ammonia:water=40:40:7:16 (v/v). A solution (2%) of ninhydrin in acetone can be used as a visualizing reagent.

The composition of the fermentation medium (g/l) is as follows:

| | |
|---|---|
| Glucose | 40.0 |
| (NH₄)₂SO₄ | 16.0 |
| K₂HPO₄ | 0.1 |
| MgSO₄•7H₂O | 1.0 |
| FeSO₄•7H₂O | 0.01 |
| MnSO₄•5H₂O | 0.01 |
| Thiamine HCl | 0.0002 |
| Yeast extract | 2.0 |
| Tyrosine | 0.125 |
| CaCO₃ | 20.0 |

Glucose and magnesium sulfate are sterilized separately. CaCO₃ is sterilized by dry-heat at 180° C. for 2 hours. pH is adjusted to 7.0.

### Example 9

### Production of L-Tryptophan by E. Coli SV164 (pGH5)-ΔNac

To test the effect of inactivation of the nac gene on L-tryptophan production, DNA fragments from the chromosome of the above-described E. coli MG1655 Δnac::cat can be transferred to the tryptophan-producing E. coli strain SV164 (pGH5) by P1 transduction (Miller, J. H. (1972) Experiments in Molecular Genetics, Cold Spring Harbor Lab. Press, Plainview, N.Y.) to obtain the strain SV164(pGH5)-Δnac. The strain SV164 has the trpE allele encoding anthranilate synthase free from feedback inhibition by tryptophan. The plasmid pGH5 harbors a mutant serA gene encoding phosphoglycerate dehydrogenase free from feedback inhibition by serine. The strain SV164 (pGH5) is described in detail in U.S. Pat. No. 6,180,373.

Both strains, SV164(pGH5)-Δnac and SV164(pGH5), can be cultivated with shaking at 37° C. for 18 hours in a 3 ml of nutrient broth supplemented with 20 μg/ml of tetracycline (marker of pGH5 plasmid). 0.3 ml of the cultures can be inoculated into 3 ml of a fermentation medium containing tetracycline (20 μg/ml) in 20×200 mm test tubes, and cultivated at 37° C. for 48 hours with a rotary shaker at 250 rpm. After cultivation, the amount of tryptophan, which accumulates in the medium can be determined by TLC as described in Example 8. The fermentation medium components are set forth in Table 3, but should be sterilized in separate groups A, B, C, D, E, F, and H, as shown, to avoid adverse interactions during sterilization.

TABLE 3

| Groups | Component | Final concentration, g/l |
|---|---|---|
| A | KH₂PO₄ | 1.5 |
| | NaCl | 0.5 |
| | (NH₄)₂SO₄ | 1.5 |
| | L-Methionine | 0.05 |
| | L-Phenylalanine | 0.1 |
| | L-Tyrosine | 0.1 |
| | Mameno (total N) | 0.07 |
| B | Glucose | 40.0 |
| | MgSO₄•7H₂O | 0.3 |
| C | CaCl₂ | 0.011 |
| D | FeSO₄•7H₂O | 0.075 |
| | Sodium citrate | 1.0 |
| E | Na₂MoO₄•2H₂O | 0.00015 |
| | H₃BO₃ | 0.0025 |
| | CoCl₂•6H₂O | 0.00007 |
| | CuSO₄•5H₂O | 0.00025 |
| | MnCl₂4H₂O | 0.0016 |
| | ZnSO₄•7 H₂O | 0.0003 |
| F | Thiamine HCl | 0.005 |
| G | CaCO₃ | 30.0 |
| H | Pyridoxine | 0.03 |

Group A has pH 7.1 adjusted by NH₄OH. Each of groups A, B, C, D, E, F and H is sterilized separately, chilled, and mixed together, and then CaCO₃ sterilized by dry heat is added to the complete fermentation medium.

### Example 10

### Production of L-Proline by E. Coli 702ilvA-ΔNac

To test the effect of inactivation of the nac gene on L-proline production, DNA fragments from the chromosome of the above-described E. coli MG1655Δnac::cat can be transferred to the proline-producing E. coli strain 702ilvA by P1 transduction (Miller, J. H. (1972) Experiments in Molecular Genetics, Cold Spring Harbor Lab. Press, Plainview, N.Y.) to obtain the strain 702ilvA-Δnac. The strain 702ilvA has been deposited in the Russian National Collection of Industrial Microorganisms (VKPM) (Russia, 117545 Moscow, 1 Dorozhny proezd, 1) on Jul. 18, 2000 under accession number VKPM B-8012 and then converted to a deposit under the Budapest Treaty on May 18, 2001.

Both E. coli 702ilvA and 702ilvA-Δnac can be grown for 18-24 hours at 37° C. on L-agar plates. Then, these strains can be cultivated under the same conditions as in Example 7.

### Example 11

### Production of L-Arginine by E. Coli 382-ΔNac

To test the effect of inactivation of the nac gene on L-arginine production, DNA fragments from the chromosome of the above-described E. coli MG1655 Δnac::cat can be transferred to the arginine-producing E. coli strain 382 by P1 transduction (Miller, J. H. (1972) Experiments in Molecular Genetics, Cold Spring Harbor Lab. Press, Plainview, N.Y.) to obtain the strain 382-Δnac. The strain 382 has been deposited in the Russian National Collection of Industrial Microorganisms (VKPM) (Russia, 117545 Moscow, 1 Dorozhny proezd, 1) on Apr. 10, 2000 under accession number VKPM B-7926 and then converted to a deposit under the Budapest Treaty on May 18, 2001.

Both strains, 382-Δnac and 382, can be each cultivated with shaking at 37° C. for 18 hours in a 3 ml of nutrient broth. 0.3 ml of the cultures can be inoculated into 3 ml of a fermentation medium in 20×200 mm test tubes, and cultivated at 32° C. for 48 hours on a rotary shaker. After the cultivation, the amount of L-arginine which accumulates in the medium can be determined by paper chromatography using following mobile phase: butanol: acetic acid:water=4:1:1 (v/v). A solution (2%) of ninhydrin in acetone can be used as a visualizing reagent. A spot containing L-arginine can be cut out, L-arginine can be eluted in 0.5% water solution of CdCl₂, and the amount of L-arginine can be estimated spectrophotometrically at 540 nm.

The composition of the fermentation medium (g/l) is as follows:

| | |
|---|---|
| Glucose | 48.0 |
| (NH4)₂SO₄ | 35.0 |
| KH₂PO₄ | 2.0 |
| MgSO₄•7H₂O | 1.0 |
| Thiamine HCl | 0.0002 |
| Yeast extract | 1.0 |
| L-isoleucine | 0.1 |
| CaCO₃ | 5.0 |

Glucose and magnesium sulfate are sterilized separately. CaCO₃ dry-heat sterilized at 180° C. for 2 hours. pH is adjusted to 7.0.

While the invention has been described in detail with reference to preferred embodiments thereof, it will be apparent to one skilled in the art that various changes can be made, and equivalents employed, without departing from the scope of the invention. All the cited references herein are incorporated as a part of this application by reference.

### INDUSTRIAL APPLICABILITY

According to the present invention, production of L-amino acid of a bacterium of the Enterobacteriaceae family can be enhanced.

1. An L-amino acid producing bacterium of the Enterobacteriaceae family, wherein said bacterium has been modified to attenuate expression of the nac gene.

2. The bacterium according to claim 1, wherein said expression of the nac gene is attenuated by inactivation of the nac gene.

3. The bacterium according to claim 1, wherein said bacterium belongs to the genus Escherichia.

4. The bacterium according to claim 1, wherein said bacterium belongs to the genus Pantoea.

5. The L-amino acid producing bacterium according to claim 1, wherein said L-amino acid is selected from the group consisting of an aromatic L-amino acid and a non-aromatic L-amino acid.

6. The L-amino acid producing bacterium according to claim 5, wherein said aromatic L-amino acid is selected from the group consisting of L-phenylalanine, L-tyrosine, and L-tryptophan.

7. The L-amino acid producing bacterium according to claim 5, wherein said non-aromatic L-amino acid is selected from the group consisting of L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine.

8. A method for producing an L-amino acid comprising:
cultivating the bacterium according to claim 1 in a medium so to produce and excrete said L-amino acid into the medium, and
collecting said L-amino acid from the medium.

9. The method according to claim 8, wherein said L-amino acid is selected from the group consisting of an aromatic L-amino acid and a non-aromatic L-amino acid.

10. The method according to claim 9, wherein said aromatic L-amino acid is selected from the group consisting of L-phenylalanine, L-tyrosine, and L-tryptophan.

11. The method according to claim 9, wherein said non-aromatic L-amino acid is selected from the group consisting of L-threonine, L-lysine, L-cysteine, L-methionine, L-leucine, L-isoleucine, L-valine, L-histidine, glycine, L-serine, L-alanine, L-asparagine, L-aspartic acid, L-glutamine, L-glutamic acid, L-proline, and L-arginine.


SEQUENCE LISTING (6 sequences)

SEQ ID NO: 1 - DNA, 918 bp, Escherichia coli
  CDS (1)..(918)
atgaacttcagacgcctgaaatacttcgtaaaaattgtagatattggtagcctgacccaggctgctgaagtattgcatatcgcacaaccagcgctcagccagcaggttgccacactggaaggtgagttaaatcaacaacttttgatccgtacaaagcggggcgttacaccaacagacgccggaaaaattctctatacccatgcgcgggccattttacgtcagtgtgaacaggcccaactggcggtgcataacgttggtcaggcattatcggggcaagtctcgattggctttgcaccaggaaccgctgcgtcatccatcaccatgcccttattacaggcggttcgcgctgaatttccggagatcgttatctatcttcatgaaaatagtggtgcagtgcttaacgaaaaattgataaatcaccaactcgatatggcggtgatttatgagcattcccctgtggctggtgtatccagtcaggctttgctgaaagaagatctttttctggtaggaactcaagattgcccggggcaaagcgttgatgtgaatgctattgcgcaaatgaacctctttctccccagtgattacagtgctattagacttcgtgttgatgaggctttttccctacggcgactcacggcaaaagttattggtgaaattgagtctattgccacgcttaccgcagcgattgccagcggcatgggcgttgcagtattacccgaatcggccgcgcgttcgttatgtggcgcagtaaatgggtggatgtcacgcattaccactccttccatgagtctctctttgtcattaaatttacccgccagagcgaacttatcgccacaggcacaggcagtgaaagagttgttaatgtcagtgattagttctccagtgatggaaaaaaggcagtggcaattggtgagctaa

SEQ ID NO: 2 - protein, 305 aa, Escherichia coli
MNFRRLKYFVKIVDIGSLTQAAEVLHIAQPALSQQVATLEGELNQQLLIRTKRGVTPTDAGKILYTHARAILRQCEQAQLAVHNVGQALSGQVSIGFAPGTAASSITMPLLQAVRAEFPEIVIYLHENSGAVLNEKLINHQLDMAVIYEHSPVAGVSSQALLKEDLFLVGTQDCPGQSVDVNAIAQMNLFLPSDYSAIRLRVDEAFSLRRLTAKVIGEIESIATLTAAIASGMGVAVLPESAARSLCGAVNGWMSRITTPSMSLSLSLNLPARANLSPQAQAVKELLMSVISSPVMEKRQWQLVS

SEQ ID NO: 3 - DNA, 57 bp, Artificial
  primer
atgaacttcagacgcctgaaatacttcgtaaaaatttagtaagccagtatacactcc

SEQ ID NO: 4 - DNA, 57 bp, Artificial
  primer
ccaattgccactgccttttttccatcactggagaacttaagggcaccaataactgcc

SEQ ID NO: 5 - DNA, 21 bp, Artificial
  primer
ggcttttttgaatttggctcc

SEQ ID NO: 6 - DNA, 21 bp, Artificial
  primer
catcccgatataacacttagc